(19) United States
(12) Patent Application Publication
(10) Pub. No.: US 2022/0118314 A1
(43) Pub. Date: Apr. 21, 2022

(54) Method and Device for Measuring Forces

(71) Applicant: KISTLER HOLDING AG, WINTERTHUR (CH)

(72) Inventors: Katharina Buechli, Boezen (CH); Dominik Jenni, Zuerich (CH); Daniel Roetenberg, Ottikon bei Kemptthal (CH); Andrej Panjan, Gradac (SI); Nejc Sarabon, Kranj (SI)

(21) Appl. No.: 17/504,783

(22) Filed: Oct. 19, 2021

(30) Foreign Application Priority Data
Oct. 20, 2020 (EP) 20202759.5

(51) Int. Cl. A63B 24/00; A63B 71/06

(52) U.S. Cl. CPC A63B 24/0062; A63B 71/0622; A63B 2220/53; A63B 2220/833; A63B 2220/62; A63B 2214/00; A63B 2220/05; A63B 2225/50; A63B 2225/20; A63B 2220/40; A63B 2244/08; A63B 2220/807

(57) ABSTRACT

A method for measuring the force generated by at least one athlete jumping on a measuring platform includes generating exercise data for jumping exercises. The exercise data is presented to the athlete. As the athlete performs jumping exercises on the measuring platform, a jumping force of the jumping exercises is measured and recorded as measurement data for the measured jumping force. The measurement data is sent to a data processing system that evaluates the measurement data and generates performance data from the measurement data. Expert data is determined from the performance data, and either the performance data or the expert data or both, are outputted to the athlete or to someone monitoring the performance of the athlete.

### FIELD OF THE INVENTION

The present invention relates to a method and a device for measuring forces involved from the jumping of an athlete.

### BACKGROUND

DE10040623A1, which corresponds to applicant's commonly owed U.S. Pat. No. 6,389,894 to Calame, which patent is hereby incorporated herein in its entirety by this reference for all purposes, discloses a method for measuring the jumping force of an athlete. For this purpose, the athlete gets onto a measuring platform that is equipped with a plurality of force sensors. The athlete performs vertical jump exercises on the measuring platform. For each jump, the measuring platform measures a vertical jumping force of the athlete and generates corresponding measurement data. The measurement data is transmitted to a data processing system that evaluates the measurement data. The result of the evaluation is the so-called performance diagnostics. Performance diagnostics includes performance information on the jumping height of the athlete, the athlete's velocity during the jumps, the athlete's force during the jumps, and so on.

Such performance diagnostics are of considerable importance in sports and medicine. For example, they provide information with respect to a performance state of the athlete. The performance state indicates how well qualities such as strength, endurance, speed, coordination, and agility are developed in the athlete.

Measuring the jumping force of one athlete typically takes about 6 minutes. In the exercises, the athlete performs solo jumps or multiple jumps. A single jump is carried out in a solo jump, while multiple jumps consist of a sequence of jumps such as triple jumps, quintuple jumps and so on performed within a time interval. The athlete repeats the jumping exercises several times. Since with this method the measuring platform can only be used by one athlete at a time, the other athletes must wait in the meantime. For this reason, it will take 2 hours to perform a jumping force measurement of a team of twenty athletes. To keep their waiting times as brief as possible, the athletes must adhere to a strict time schedule for the jumping force measurement, and adherence to such a strict schedule causes stress for the athletes and their trainers. However, apart from the desirability of reducing stress generally for better health, the jumping force measurement is performed more accurately and reliably when the athletes are free of stress caused by waiting anxiety.

However, performance diagnostics is not only important for the athlete himself or herself but also for his or her coach or supervisor. Generally, the supervisor is present at the measuring platform to instruct the jumping exercises. For a team of twenty athletes, the supervisor would need to be present at the platform for 2 hours. Thus, the supervisor desires to reduce the time the supervisor needs to instruct the jumping exercises.

The performance diagnostics is output on a screen of the data processing system. The measuring platform and the data processing system are located in close spatial proximity to one another. This means that even after the jumping exercises have ended, the athletes and the coach must stay close to the data processing system to learn about the performance diagnostics. This requires additional time. Also in this case, the athletes and the supervisor do not wish to wait a long time to get information about the performance diagnostics.

Performance diagnostics also includes the athlete's historical performance information. The athlete's historical performance information informs the athlete and coach with respect to the development of the performance state of the athlete at the different points in time when the performance diagnostics are generated. It is the main goal of the athletes and the coach, particularly in competitive sports, to optimize the performance state of the athlete. The jumping exercises are generally designed for the athlete to have an optimal performance state on a certain date. The athletes and the coach want to design the jumping exercises specifically for reaching that goal and, therefore, are also interested in an interpretation of the performance diagnostics.

### OBJECTS AND SUMMARY OF THE INVENTION

Thus, it is one object of the present invention to provide a method and device that allow for carrying out the jumping force measurement free of stress for the athletes. It is another object of the invention to provide a method and device that reduce the amount of time the coach needs to instruct the jumping exercises. Furthermore, a further object of the invention relates to obtaining information regarding the performance diagnostics quickly by the athletes and the coach. An additional object of the invention is to suggest a method and a device that assist the athletes and the coach in interpreting the performance diagnostics.

At least one of these objects has been achieved by the features described hereinafter.

The invention relates to a method for measuring the force of at least one athlete by using a measuring platform; wherein in a first step exercise data for jumping exercises are generated by means of a data processing system which transmits the exercise data to a first computer means; wherein in a second step jumping exercises are instructed for which purpose said athlete uses said first computer means wherein said exercise data are output to the athlete on said first computer means; wherein the athlete performs jumping exercises on said measuring platform which measures a jumping force of said jumping exercises, which generates measurement data for said measured jumping force, and which transmits said measurement data to said data processing system; wherein in a third step the measurement data are evaluated by using the data processing system which evaluates the measurement data to give performance data, which determines expert data for said performance data, and which transmits said performance data and expert data to at least one of the following: the first computer means and a second computer means; and wherein in a fourth step said performance data and said expert data are output on at least one of the following: the first computer means and the second computer means.

The invention has the advantage that the coach no longer has to instruct the jumping exercises herself or himself which saves his or her time. In addition, the evaluated performance data are provided to the athlete at the first computer means and/or to the supervisor at the second computer means in a timely manner. In addition, expert data are determined for the performance data which expert data assist the athlete and/or the supervisor in interpreting the performance data. Thereby, the effectiveness of the jumping exercises of the athlete is greatly enhanced.

The invention also relates to a device for carrying out said method comprising a measuring platform, a data processing system and a first computer means, wherein the device further comprises a data transmission means; wherein the data processing system transmits exercise data for jumping exercises to the first computer means via said data transmission means; the measuring platform comprises a measuring platform processor configured to generate measurement data for jumping exercises that are carried out on the measuring platform; wherein the measuring platform transmits the measurement data to the data processing system via the data transmission means; wherein the data processing system comprises a main processor configured to evaluate the measurement data to calculate performance data and to determine expert data for the performance data; and wherein the data transmission means transmits the performance data and the expert data via the data transmission means to at least one of the following: the first computer means and a second computer means.

### BRIEF DESCRIPTION OF THE SEVERAL VIEWS OF THE DRAWINGS

In the following, the invention is explained in more detail by way of example referring to the figures in which

FIG. 1 is a flow chart showing steps I to IV of a method according to an embodiment of the invention;

FIG. 2 is a schematic representation of a device 10 for carrying out the method according to FIG. 1;

FIG. 3 is a flow chart showing a schematic representation of first sub-steps XI to XIII of a first step I of the method according to FIG. 1;

FIG. 4 is a schematic representation of an output of exercise data D5 provided in the first step I of the method according to FIG. 3 on a first output means AU5;

FIG. 5 is a schematic representation of an output of exercise data D5 provided in the first step I of the method according to FIG. 3 on a second output means AU6;

FIG. 6 is a flow chart showing a schematic representation of second sub-steps XXI to XXIII of a second step II of the method according to FIG. 1;

FIG. 7 is a flow chart showing a schematic representation of third sub-steps XXXI to XXXIV of a third step III of the method according to FIG. 1;

FIG. 8 is a flow chart showing a schematic representation of fourth sub-steps XLI to XLIII of a fourth step IV of the method according to FIG. 1;

FIG. 9 is a schematic representation of an output of performance data D10 and expert data D4 determined in the third step III of the method according to FIG. 7 on the first output means AU5;

FIG. 10 is a schematic representation of an output of performance data D10 and expert data D4 determined in the third step III of the method according to FIG. 7 on the second output means AU6;

FIG. 11 is a graph showing an output of a fifth expert information E5 determined in the third step III of the method according to FIG. 7;

FIG. 12 is a schematic representation of an output of exercise data D5 provided in the first step I of the method according to FIG. 3 and of performance data D10 and expert data D4 determined in the third step III of the method according to FIG. 7 on the first output means AU5; and

FIG. 13 is a schematic representation of an output of exercise data D5 provided in the first step I of the method according to FIG. 3 on a second output means AU6.

Throughout the figures, identical reference numerals denote similar features.

### DETAILED DESCRIPTION OF EXEMPLARY EMBODIMENTS OF THE INVENTION

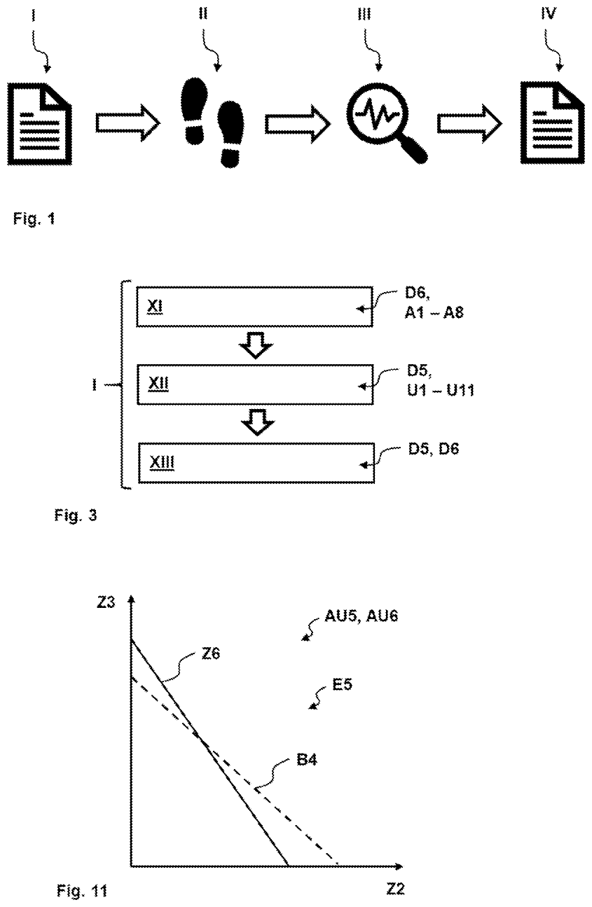

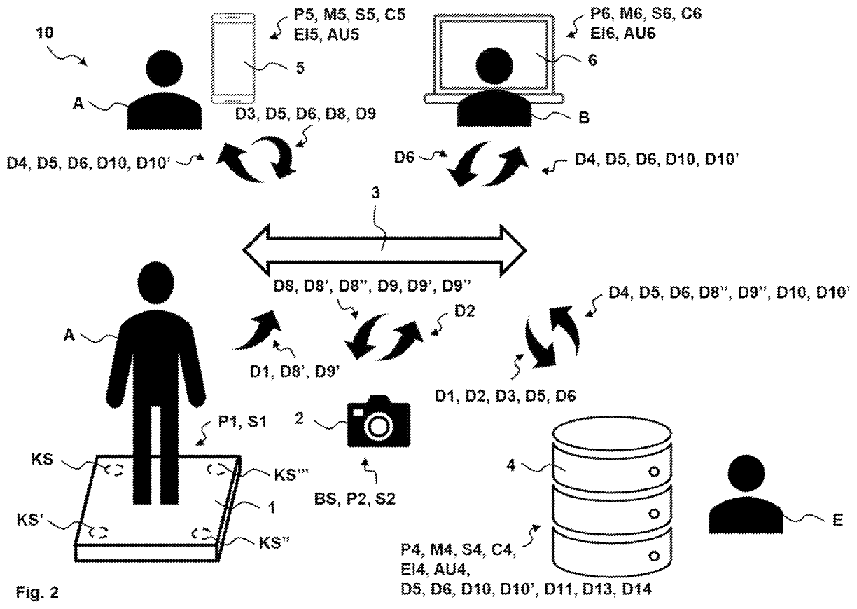

FIG. 1 is a flow chart showing a first step I of the method, a second step II of the method, a third step III of the method and a fourth step IV of the method. FIG. 2 is a schematic representation of a device 10 for carrying out the method.

For the purposes of the present invention, a distinction is made between digital data and information. Digital data is provided to a machine such as a processor, a computer program, a storage medium, etc. while information is provided to a human such as an athlete, a coach, etc. Digital data is readable and processable by the machine only, while information is understood and used by human individuals only.

As schematically shown in FIG. 2, the device 10 comprises at least one measuring platform 1. The measuring platform 1 comprises a standing platform desirably having an area of only just 1 m². The athlete A gets onto the standing platform to perform the jumping exercises. The measuring platform 1 is a diagnostic tool for determining the performance state of the athlete A. The measuring platform 1 is not a training tool for optimizing the performance state of the athlete A. Athlete A uses known training tools such as strength training machines for training strength, treadmills and bicycles for training endurance and speed, etc. to optimize his or her performance state. The measuring platform 1 comprises a plurality of force sensors KS, KS′, KS″, KS′″ and at least one measuring platform processor P1. The force sensors KS, KS′, KS″, KS′″ measure a jumping force for each vertical jump of the athlete A and accordingly generate an output signal in the form of digital data for each measurement. The force sensors KS, KS′, KS″, KS′″ are connected to at least one measuring platform processor P1, which receives the digital output signals for the jumping force measurements and is configured with algorithms for processing the signals to yield information.

The jumping force is a ground reaction force. According to Newton's third law, the ground reaction force is the force exerted by the ground onto the athlete A being in contact with the ground. When athlete A is at rest in a standing position the ground reaction force corresponds to the weight of athlete A. During movements, the ground reaction force changes due to acceleration forces. Thus, the force acting onto athlete A during running is equal to two to three times the force that corresponds to athlete A's weight. A measuring range of the force sensors KS, KS′, KS″, KS′″ is from 0 to 10 kN. A measuring frequency of the force sensors KS, KS′, KS″, KS′″ is 500 Hz. Each of the force sensors KS, KS′, KS″, KS′″ desirably includes one or more piezoelectric crystals that generate analog electrical signals proportional to an incident force and analog-to-digital converters that transform the analog signals into digital signals. Thus, the measuring platform processor P1 is configured to generate digital measurement data D1 for the measured jumping force. According to an embodiment of the present invention, an athlete may also perform the jumping exercises on two measuring platforms simultaneously. The measuring platforms will be identical. The athlete may stand with a left leg on a first measuring platform while the athlete stands with a right leg on a second measuring platform. Both measuring platforms measure a jumping force and generate measurement data D1 for the measured jumping force independently of one another.

As schematically shown in FIG. 2, the device 10 comprises at least one camera 2. The camera 2 comprises at least one image sensor BS and at least one camera processor P2. The image sensor BS captures images of the athlete A while the athlete A performs the jumping exercises. A desired acquisition frequency of the image sensor BS is 100 frames per second (fps). The camera processor P2 is configured to generate image data D2 for the captured images.

As schematically shown in FIG. 2, the device 10 comprises at least one data transmission means 3 for transmitting digital data. The digital data are transmitted along a transmission path between interfaces. The interfaces are configured to perform a transmission and reception of digital data. The digital data are measurement data D1, image data D2, expert data D4, exercise data D5, and athlete data D6. In FIG. 2, the digital data are schematically shown as curved arrows. The digital data are transmitted by radio or cable according to a protocol. According to the protocol, the interfaces are unequivocally identified by identification numbers. The data transmission means 3 may be a Universal Serial Bus (USB), Bluetooth, Ethernet, Internet, a wireless local area network (WLAN), a fixed network, etc. The measuring platform 1 comprises at least one measuring platform interface S1. Furthermore, the camera 2 also comprises at least one camera interface S2.

As schematically shown in FIG. 2, the device 10 comprises at least a data processing system 4. The data processing system 4 is used by at least a developer E such as a technologist, etc. The data processing system 4 may be located anywhere in the world. The data processing system 4 comprises at least a main processor P4, at least a main storage medium M4 configured for electronically storing digital data, and at least a main interface S4 for the transmission and reception of digital data. At least a main computer program C4 is loaded into the processor P4 and is executed by the main processor P4. The main computer program C4 is executed by the main processor P4 to execute the first step I of the method and the third step III of the method schematically shown in FIG. 1. The data processing system 4 includes at least a main input means E14 such as a keyboard, a microphone, a camera, etc. The data processing system 4 comprises at least a main output means AU4 such as a screen, a loudspeaker, etc.

As schematically shown in FIG. 2, the device 10 comprises at least a first computer means 5 and at least a second computer means 6. The first computer means 5 is used by the athlete A. Athlete A has at least a supervisor B such as a coach, a therapist, etc. The second computer means 6 is used by the supervisor B. Preferably, the first and second computer means 5, 6 are used at the location of the force measurement. Each of the first computer means 5 and the second computer means 6 may be a personal computer (PC), a laptop, a smartphone, a smartwatch, etc. In the example shown in FIG. 2, the first computer means 5 is schematically represented as a smartphone, while the second computer means 6 is schematically represented as a Personal Computer (PC).

As schematically shown in FIG. 2, the first computer means 5 comprises at least a first processor P5, at least a first storage medium M5 configured for electronically storing digital data, and at least a first interface S5 for the transmission and reception of digital data. At least a first computer program C5 is loaded into the first processor P5 and is executed by the first processor P5. The first computer program C5 is executed by the first processor P5 of the first computer means 5 to execute the second step II of the method and the fourth step IV of the method schematically shown in FIG. 1. The first computer means 5 comprises at least a first input means E15 such as a keyboard, a touch screen, a microphone, a camera, etc. The first computer means 5 comprises at least a first output means AU5 such as a touch screen, a loudspeaker, etc.

The second computer means 6 comprises at least a second processor P6, at least a second storage medium M6 configured for electronically storing digital data, and at least a second interface S6 for the transmission and reception of digital data. At least a second computer program C6 is loaded into the second processor P6 and is executed by the second processor P6. The second computer program C6 is executed by the second processor P6 of the second computer means 6 to execute the fourth step IV of the method schematically shown in FIG. 1. The second computer means 6 comprises at least a second input means E16 such as at least a keyboard, a microphone, a camera, etc. The second computer means 6 comprises at least a second output means AU6 such as a screen, a loudspeaker, etc.

### First Step I

In the first step I of the method schematically shown in FIG. 1, exercise data D5 for jumping exercises are generated by the data processing system 4. FIG. 3 is a detailed representation of the first step I, which includes an eleventh sub-step XI, a twelfth sub-step XII, and a thirteenth sub-step XIII.

In accordance with the eleventh sub-step XI of the first step I of the method schematically shown in FIG. 1, at least an athlete information A1 to A8 is provided. Each athlete A can be unequivocally identified by means of the athlete information A1 to A8. The athlete information A1 to A8 is newly generated for an athlete A who is coached for the first time by the coach or supervisor B. The athlete information A1 to A8 is an alphanumeric string, an image, a graph, etc. Athlete information A1 to A8 may be provided in a variety of ways:

For example, the athlete information A1 to A8 may be entered into the second computer means 6 for which purpose the supervisor B uses the second input means E16. The second computer means 6 converts the athlete information A1 to A8 into athlete data D6, which may be sent by second computer means 6 via the second interface S6 to the data processing system 4 as the athlete data D6. The athlete data D6 may be received by the data processing system 4 via the main interface S4 and may be stored in the main storage medium M4. Additionally, the athlete information A2 to A8 may also be updated by athlete A in this way.

However, the athlete information A1 to A8 may also be input into the first computer means 5 via the first input means E15 by the athlete A. The first computer means 5 converts the athlete information A1 to A8 into athlete data D6 and transmits the athlete data D6 via the first interface S5 to the data processing system 4. The athlete data D6 may be received by the data processing system 4 via the main interface S4 and may be stored electronically in the main storage medium M4.

Furthermore, in accordance with the present invention, the device 10 is configured so that the developer E can enter the athlete information A1 to A8 into the data processing system 4 via the main input means E14, whereupon the data processing system 4 converts the athlete information A1 to A8 into athlete data D6, and electronically stores the athlete data D6 in the main storage medium M4.

Examples of athlete information A1 through A8 are:
A first athlete information A1 is an identification number uniquely assigned to the athlete A. The first athlete information A1 is generated in the beginning and is not changed afterwards.
A second athlete information A2 is a name of athlete A.
A third athlete information A3 is a gender of athlete A.
A fourth athlete information A4 is an age of athlete A.
A fifth athlete information A5 is a body height of athlete A.
A sixth athlete information A6 is a weight of athlete A.
A seventh athlete information A7 is a type of sport performed by athlete A.
An eighth athlete information A8 is a freely selectable sports characteristic of athlete A, such as “striker”, “left-footed player”, “junior”, and the like. Additional characteristics also can be included and associated with the athlete A having the unique identification number A1.

In accordance with the twelfth sub-step XII of the first step I of the method schematically shown in FIG. 1, exercise information U1 to U11 is generated. The generation of exercise information U1 to U11 may be carried out by the developer E who enters exercise information U1 to U11 via the main input means E14 into the data processing system 4, which converts the exercise information U1 to U11 into exercise data D5 and electronically stores the exercise data D5 in the main storage medium M4 as the exercise data D5. The exercise information U1 to U11 is entered in the beginning and may be retrieved afterwards as the exercise data D5 any number of times from the main storage medium M4.

The exercise information U1 to U11 includes information regarding jumping exercises for strength, endurance, velocity, coordination, and agility of athlete A. By the exercise information U1 to U11, athlete A is provided with information as to which jumping exercises to perform how many times and in which order as well as the break intervals which have must be kept in between the exercises. In addition, by means of the exercise information U1 to U11, athlete A's coach B is kept informed about which jumping exercises athlete A will perform or has performed how many times, in which order and which break intervals athlete A observed in between the exercises. The exercise information U1 to U11 can take the form of any of an alphanumeric string, an image, etc.

Examples of exercise information U1 to U11 are:
A first exercise information U1 indicates the exercise date.
A second exercise information U2 indicates an order of the jumping exercises.
A third exercise information U3 indicates a break interval between the jumping exercises.
A fourth exercise information U4 indicates a number of vertical solo jumps to be performed from an upright position, either one-legged on the left or the right leg or on both legs (countermovement jump).
A fifth exercise information U5 indicates a number of vertical solo jumps to be performed from a squat position, either one-legged on the left or the right leg or on both legs (squat jump).
A sixth exercise information U6 indicates a number of vertical solo jumps to be performed from a predefined drop height (drop jump).
A seventh information U7 indicates a number of vertical solo jumps to be performed from an upright position, either one-legged on the left or the right leg or on both legs (countermovement jump) while carrying a predefined additional weight.
An eighth exercise information U8 indicates a number of vertical solo jumps to be performed from a squat position, either one-legged on the left or the right leg or on both legs (squat jump) while carrying a predefined additional weight.
A ninth exercise information U9 indicates a time period for performing multiple jumps.
A tenth exercise information U10 indicates a number of squats.
An eleventh exercise information U11 indicates a time period for holding a balance position, either one-legged on the left or the right leg or on both legs with optional handicaps such as eyes closed, head tilted backwards, etc.

In accordance with the thirteenth sub-step XIII of the first step I of the method schematically shown in FIG. 1, the athlete data D6 and the exercise data D5 are provided to the athlete A and the coach B. For this purpose, the athlete data D6 and the exercise data D5 are sent by the data processing system 4 via the main interface S4 to the first computer means 5. The first computer means 5 receives the athlete data D6 and the exercise data D5 via the first interface S5. The first computer means 5 electronically stores the athlete data D6 and the exercise data D5 in the first storage medium S5. In addition, the athlete data D6 and the exercise data D5 are sent by the data processing system 4 via the main interface S4 to the second computer means 6. The second computer means 6 receives the athlete data D6 and the exercise data D5 via the second interface S6. The second computer means 6 electronically stores the athlete data D6 and the exercise data D5 in the second storage medium S6.

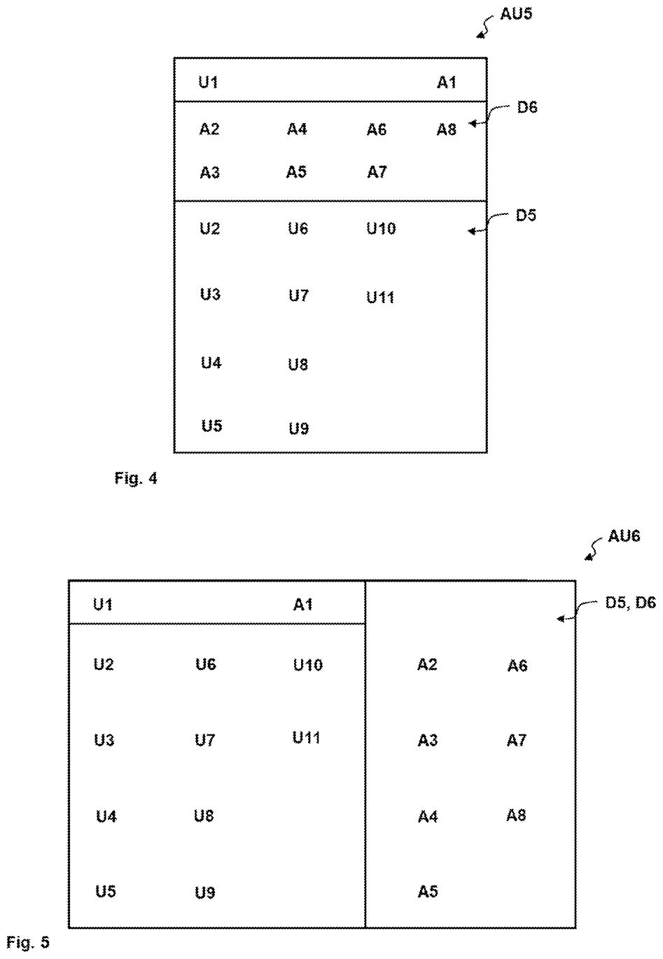

FIG. 4 is a schematic representation of the athlete data D6 and the exercise data D5 that are output by the first computer program C5 on the first output means AU5 of the first computer means 5. The first output means AU5 is for example a screen with 5.5″ diagonal. The entirety of the athlete data D6 is output as the athlete information A1 to A8. The entirety of the exercise data D5 is output as the exercise information U1 to U11. In this manner, athlete A receives the relevant information in a single output. However, athlete A may also share access to the first output means AU5 with the supervisor B to simultaneously obtain knowledge of the relevant information in a timely manner.

FIG. 5 is a schematic representation of the athlete data D6 and the exercise data D5 that are output by the second computer program C5 on the second output means AU6 of the second computer means 6. The second output means AU6 is for example a screen with 22″ diagonal. The entirety of the athlete data D6 is output as the athlete information A1 to A8. The entirety of the exercise data D5 is output as the exercise information U1 to U11. In this manner, the supervisor B obtains the relevant information in a single output. However, the supervisor B may also share access to the second output means AU6 with the athlete A to simultaneously obtain knowledge of the relevant information in a timely manner.

The supervisor B and the athlete A discuss the performance of the jumping exercises to be performed on the exercise date U1. This communication may be via known communication means such as telephone, short messaging service (SMS), electronic mail (email), and the like. The result of the communication is a selection of exercise data D5 together with at least an exercise information U1 to U11 that provides information regarding jumping exercises to be performed by athlete A on the exercise date U1.

### Second Step II

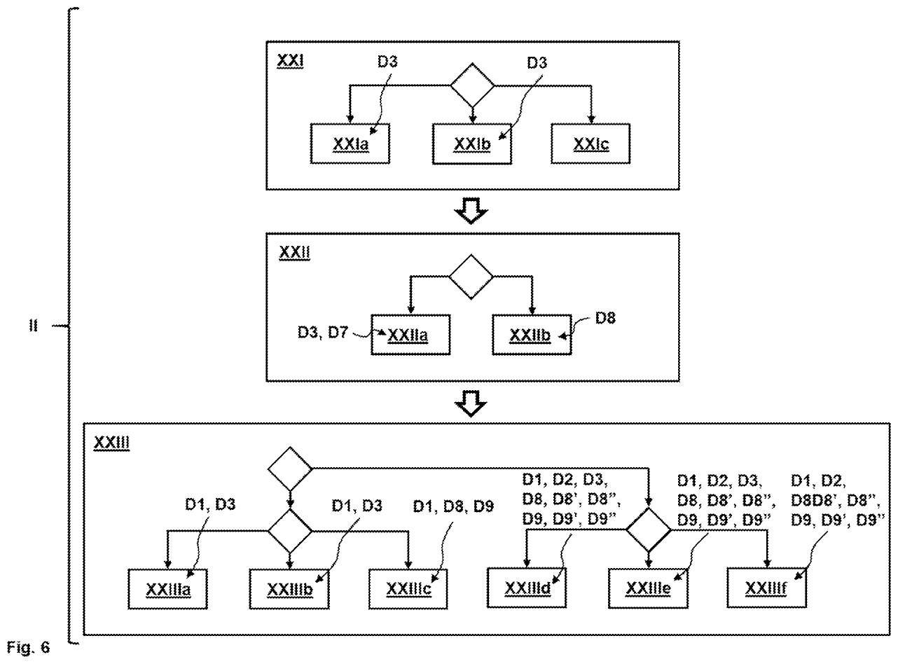

In accordance with the second step II of the method schematically presented in FIG. 1, an instruction of jumping exercises is carried out by the athlete A with the unique identification A1. It is the athlete A who is instructed how to perform the jumping exercises. In the example according to FIG. 2, athlete A performs the jumping exercises on the measuring platform 1. FIG. 6 is a detailed schematic representation of the second step II, which desirably includes a twenty-first sub-step XXI, a twenty-second sub-step XXII, and a twenty-third sub-step XXIII.

In accordance with the present invention and schematically shown in FIG. 6, there are three alternative ways for performing the twenty-first sub-step XXI. These three alternatives include: a first twenty-first alternative step XXIa or a second twenty-first alternative step XXIb or a third twenty-first alternative step XXIc.

According to the first twenty-first alternative step XXIa, athlete A goes to the measuring platform 1. The first computer means 5 of the athlete A is configured to identify the measuring platform 1. For example, the measuring platform 1 has an identification number such as a bar code, a quick response (QR) code, etc. by which the measuring platform 1 may be uniquely identified. The first input means EI5 of the first computer means 5 takes the form of a camera that Athlete A uses to scan the identification number of the measuring platform 1. The first computer program C5 of the first computer means 5 recognizes the scanned identification number and uses the recognized identification number of the measuring platform 1 and an identification number of the first computer means 5 for generating identification data D3 for uniquely identifying athlete A. The first computer means 5 transmits the identification data D3 via the first interface S5 to the main interface S4 of the data processing system 4 so that the identification data D3, which identifies athlete A, are received by the main interface S4 of the data processing system 4. The main computer program C4 of the data processing system 4 is configured to read the identification data D3.

According to the second twenty-first alternative step XXIb, the first computer means 5 of the athlete A is automatically identified as soon as it is present in the proximity of the measuring platform 1. In the context of the present invention the noun “proximity” refers to a distance of less than 30 m. For example, the first interface S5 of the first computer means 5 transmits identification data D3, which includes an identification number of the first computer means 5, for uniquely identifying athlete A in regular time intervals. The identification data D3 are received by the main interface S4 of the data processing system 4. The main computer program C4 of the data processing system 4 is configured to read the identification data D3.

According to the third twenty-first alternative step XXIc, athlete A goes to the measuring platform 1.

In accordance with the twenty-second sub-step XXII of the second step II of the method schematically presented in FIG. 1, athlete A is present at the measuring platform 1. In this case, the twenty-second sub-step XXII consists of two alternatives: a first twenty-second alternative step XXIIa or a second twenty-second alternative step XXIIb.

According to the first twenty-second alternative step XXIIa, the main computer program C4 of the data processing system 4 determines whether identification data D3 have been entered and whether measurement data D1 are currently being received from the main interface S4 of the data processing system 4, i.e. whether measuring platform 1 is occupied or not. If identification data D3 have been entered and no measurement data D1 are currently being received from the main interface S4, then the main computer program C4 is configured to generate exercise time data D7 for an allocated exercise time. The identification data D3 include an identification number of the first computer means 5 of the athlete A. The data processing system 4 transmits the allocated exercise time as the exercise time data D7 via the main interface S4 to the first computer means 5 that has been identified by the identification number, and the computer means 5 receives the exercise time data D7 via the first interface S5. The exercise time data D7 are output to athlete A on the first output means AU5 of the first computer means 5 as the allocated exercise time when the measuring platform 1 will be vacant and ready for athlete A to use the measuring platform 1. Athlete A acknowledges the allocated exercise time and is free to otherwise occupy the athlete's time until the allocated exercise time starts and the athlete is to get onto the measuring platform 1. The assignment of an allocated exercise time has the advantage that athlete A does not need to wait around nearby until the measuring platform 1 becomes vacant for the chance to use the measuring platform 1. In this way, the stress associated with waiting around for an opportunity to use the measuring platform 1 is eliminated, and accordingly the stress of athlete A is reduced when athlete A uses the measuring platform 1.

As schematically shown in FIG. 6, according to the second twenty-second alternative step XXIIb, athlete A gets onto the measuring platform 1 when the measuring platform 1 is vacant. Athlete A confirms his or her readiness to start the jumping exercises by the start signal. He or she operates the first input means EI5 that has the form of a key, to enter the start signal into the first computer means 5, which electronically sends the start signal as first start data D8 to the first interface S5 and on to the data processing system 4 where the first start data D8 are received via the main interface S4. The first start data D8 also may include an identification number of the first computer means 5 for uniquely identifying athlete A. The main computer program C4 of the data processing system 4 electronically reads the first start data D8.

As schematically shown in FIG. 6, in the twenty-third sub-step XXIII, athlete A performs jumping exercises on the measuring platform 1. As schematically shown in FIG. 5, the jumping exercises are performed according to instructions given by the exercise information U2 to U11. For this purpose, the exercise data D5 are output on the first output means AU5 as the exercise information U2 to U11. Thus, the second exercise information U2 is output first on the first output means AU5 and executed by athlete A as jumping exercises, for example. Afterwards, the third exercise information U3 is output and executed by athlete A as jumping exercises. Then, the fourth exercise information U4 is output and executed by athlete A as jumping exercises. In addition, the fifth exercise information U5 is output and executed by athlete A as jumping exercises. Thereafter, the sixth exercise information U6 is output and executed by athlete A as jumping exercises. Furthermore, the seventh exercise information U7 is also output and executed by athlete A as jumping exercises. Finally, the eighth exercise information U8 is output and executed by athlete A as jumping exercises. The measuring platform processor P1 electronically generates measurement data D1 of the jumping force measured, and these measurement data D1 are sent electronically as the measurement data D1 via the measuring platform interface S1 to the data processing system 4 where the measurement data D1 are received via the main interface S4. The measurement data D1 are stored electronically in the main storage medium M4 of the data processing system 4.

As schematically shown in FIG. 6, the twenty-third sub-step XXIII comprises six alternatives: a first twenty-third alternative step XXIIIa or a second twenty-third alternative step XXIIIb or a third twenty-third alternative step XXIIIc or a fourth twenty-third alternative step XXIIId or a fifth twenty-third alternative step XXIIIe or a sixth twenty-third alternative step XXIIIf.

The jumping exercises may be performed by the athlete A with or without images of athlete A being captured by the camera 2 shown schematically in FIG. 2. In the first twenty-third alternative step XXIIIa, in the second twenty-third alternative step XXIIIb, and in the third twenty-third alternative step XXIIIc, the camera 2 does not capture any images of athlete A during the jumping exercises performed by the athlete A. In the fourth twenty-third alternative step XXIIId, in the fifth twenty-third alternative step XXIIIe, and in the sixth twenty-third alternative step XXIIIf, the camera 2 does capture images of athlete A during the performance of the jumping exercises by the athlete A. The camera 2 may automatically start capturing images of athlete A after the camera 2 receives the start data D8, D8′, D8″, and the camera 2 may stop capturing images of athlete A after the camera 2 receives the stop data D9, D9′, D9″.

Thus, the first computer means 5 may transmit first start data D8 electronically via the first interface S5 to the camera interface S2 of the camera 2 as soon as athlete A has confirmed his or her readiness to start the jumping exercises by the start signal. Additionally, the first computer means 5 may transmit first stop data D9 electronically via the first interface S5 to the camera interface S2 of the camera 2 as soon as athlete A has confirmed the end of the jumping exercises by a stop signal.

Furthermore, the measuring platform 1 may also transmit second start data D8′ via the measuring platform interface S1 to the camera interface S2 of the camera 2 as soon as the measurement data D1 are generated. Additionally, the measuring platform 1 may transmit second stop data D9′ via the measuring platform interface S1 to the camera interface S2 of the camera 2 as soon as no further measurement data D1 are generated.

Alternatively, the data processing system 4 may also transmit third start data D8″ via the main interface S4 to the camera interface S2 of the camera 2 as soon as the athlete A has acknowledged his or her readiness to start the jumping exercises by the start signal. Additionally, the data processing system 4 may transmit third stop data D9″ via the main interface S4 to the camera interface S2 of the camera 2 as soon as athlete A has acknowledged the end of the jumping exercises by a stop signal.

The captured images are sent electronically as the image data D2 via the camera interface S2 to the data processing system 4 where the image data D2 are received by the main interface S4. The image data D2 are stored electronically in the main storage medium M4 of the data processing system 4.

As schematically shown in FIG. 6, in the first twenty-third alternative step XXIIIa, athlete A performs the jumping exercises on the measuring platform 1 after the transmission of identification data D3. Measurement data D1 of the measuring platform 1 that are received by the data processing system 4 directly, in terms of time, after the identification data D3 are assigned by the main computer program C4 to athlete A who is uniquely identified by the identification data D3. When the data processing system 4 receives no further measurement data D1 from the measuring platform 1 for a predefined period of time, then the main computer program C4 automatically terminates the assignment of the measurement data D1 to athlete A who is uniquely identified by the identification data D3.

As schematically shown in FIG. 6, in the second twenty-third alternative step XXIIIb, athlete A performs the jumping exercises on the measuring platform 1 at the exercise time allocated to him or her by the exercise time data D7. Measurement data D1 of the measuring platform 1 that are received by the data processing system 4 directly, in terms of time, after the exercise time that was allocated by the exercise time data D7 are assigned by the main computer program C4 to athlete A who is uniquely identified by the identification data D3. When the data processing system 4 receives no further measurement data D1 from the measuring platform 1 for a predefined period of time, then the main computer program C4 automatically terminates the assignment of the measurement data D1 to athlete A who is uniquely identified by the identification data D3.

As schematically shown in FIG. 6, in the third twenty-third alternative step XXIIIc, athlete A performs the jumping exercises on the measuring platform 1 after the transmission of first start data D8. Measurement data D1 of the measuring platform 1 that are received by the data processing system 4 directly, in terms of time, after the first start data D8 are assigned by the main computer program C4 to athlete A who is uniquely identified by the first start data D8. Athlete A confirms an end of the jumping exercises by a stop signal. The athlete A desirably uses the first input means E15, which has the form of a key, to enter the stop signal electronically into the first computer means 5, which sends the first stop data D9 via the first interface S5 to the data processing system 4 where the first stop data D9 are received electronically by the main interface S4. The first stop data D9 may comprise athlete data D6 for uniquely identifying athlete A. The first stop data D9 may comprise an identification number of the first computer means 5 for uniquely identifying athlete A. The main computer program C4 electronically reads the first stop data D9. Measurement data D1 of the measuring platform 1 that the data processing system 4 receives after, in terms of time, the first stop data D9, are no longer assigned to athlete A by the main computer program C4. Confirming the end of the jumping exercises by a stop signal is optional. Alternatively, when the data processing system 4 receives no further measurement data D1 from the measuring platform 1 for a predefined period of time, then the computer program C4 automatically stops assigning the measurement data D1 to athlete A.

As schematically shown in FIG. 6, in the fourth twenty-third alternative step XXIIId, athlete A performs the jumping exercises on the measuring platform 1 after the transmission of identification data D3. Measurement data D1 of the measuring platform 1 and image data D2 of the camera 2 are received by the data processing system 4 directly, in terms of time, after the identification data D3 are assigned by the main computer program C4 to athlete A who is uniquely identified by the identification data D3. When the data processing system 4 receives no further measurement data D1 from the measuring platform 1 and no further image data 2 from the camera 2 for a predefined period of time, then the main computer program C4 automatically terminates the assignment of the measurement data D1 and the image data D2 to athlete A who is uniquely identified by the identification data D3.

As schematically shown in FIG. 6, in the fifth twenty-third alternative step XXIIIe, athlete A performs the jumping exercises on the measuring platform 1 at the exercise time allocated to her or him by the exercise time data D7. Measurement data D1 of the measuring platform 1 and image data D2 of the camera 2 are received by the data processing system 4 directly, in terms of time, after the exercise time that was allocated by the exercise time data D7 are assigned by the main computer program C4 to athlete A who is uniquely identified by the identification data D3. When the data processing system 4 receives no further measurement data D1 from the measuring platform 1 and no more image data D2 from the camera 2 for a predefined period of time, then the main computer program C4 automatically terminates the assignment of the measurement data D1 to athlete A who is uniquely identified by the identification data D3.

As schematically shown in FIG. 6, in the sixth twenty-third alternative step XXIIIf, athlete A performs the jumping exercises on the measuring platform 1 after the transmission of first start data D8. Measurement data D1 of the measuring platform 1 and image data D2 of the camera 2 are received by the data processing system 4 directly, in terms of time, after the first start data D8 are assigned by the main computer program C4 to athlete A who is uniquely identified by the first start data D8. Athlete A confirms an end of the jumping exercises by a stop signal. The Athlete A desirably uses the first input means E15, which desirably takes the form of a key, to enter the stop signal into the first computer means 5, which electronically sends the stop signal as the first stop data D9 via the first interface S5 to the data processing system 4 where the first stop data D9 are received by the main interface S4. The first stop data D9 may comprise athlete data D6 for uniquely identifying the athlete A. The first stop data D9 may comprise an identification number of the first computer means 5 for uniquely identifying the athlete A. The main computer program C4 reads the first stop data D9. Measurement data D1 of the measuring platform 1 and image data D2 of the camera 2 that are received by the data processing system 4 after, in terms of time, the first stop data D9, are no longer assigned to athlete A by the main computer program C4. Also in this case, the main computer program C4 is configured with the optional capability for electronically generating a stop signal that confirms the end of the jumping exercises. Alternatively, when the data processing system 4 no longer receives measurement data D1 from the measuring platform 1 for a predefined period of time, then the computer program C4 automatically terminates the assignment of the measurement data D1 to athlete A.

However, in consideration of this disclosure of the present invention, those skilled in the art should be empowered to implement variations of the second step II. For example, the first computer means 5 may be a smartwatch, and the exercise time data and instruction data may be output to the athlete on a first output means of the smartwatch, and the athlete may enter the start signal and the stop signal using a first input means of the smartwatch.

### Third Step III

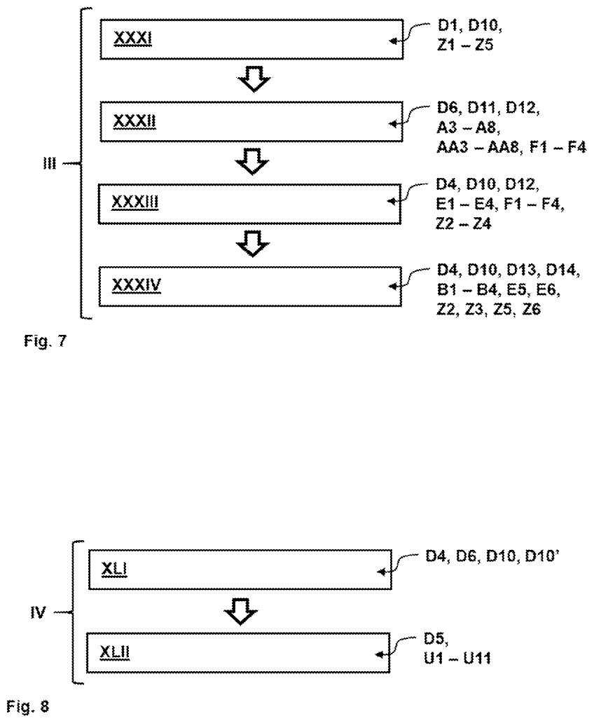

An evaluation of the measurement data D1 is carried out in the third step III, as schematically shown in FIG. 7. In the example shown in FIG. 2, the evaluation of the measurement data D1 is done by the main computer program C4 of the data processing system 4. FIG. 7 is a detailed schematic representation of the third step III that comprises a thirty-first sub-step XXXI, a thirty-second sub-step XXXII, a thirty-third sub-step XXXIII and a thirty-fourth sub-step XXXIV.

As schematically shown in FIG. 7, in the thirty-first sub-step XXXI, the main computer program C4 evaluates the measurement data D1 and generates performance data D10. An evaluation of measurement data for obtaining performance data is disclosed in DE10040623A1, which corresponds to applicant's commonly owed U.S. Pat. No. 6,389,894 to Calame, which patent is hereby incorporated herein in its entirety by this reference for all purposes. For this purpose, at least one of the following calculations of performance data D10 is carried out:

The main computer program C4 is configured to divide the measurement data D1 by the weight of athlete A and, thus, determines an acceleration. Advantageously, the main computer program C4 is configured to classify the measurement data D1 temporally in vertical single jumps or vertical multiple jumps. The main computer program C4 is configured to determine an acceleration for each of the vertical single jumps or vertical multiple jumps. The main computer program C4 is configured to determine a mean acceleration value for a plurality of vertical single jumps or vertical multiple jumps. The mean value of the accelerations is referred to as the jumping acceleration information Z1.

The main computer program C4 is configured to determine a velocity by integrating the measurement data D1 once over time. Advantageously, the main computer program C4 is configured to categorize the measurement data D1 temporally in vertical single jumps or vertical multiple jumps. The main computer program C4 is configured to determine a velocity for each of the vertical single jumps or vertical multiple jumps. The main computer program C4 is configured to determine a mean velocity value for a plurality of vertical single jumps or vertical multiple jumps. The mean value of the velocity is referred to as the jumping velocity information Z2.

The main computer program C4 is configured to determine a mean jumping force value from the measurement data D1 of a plurality of vertical single jumps or vertical multiple jumps. Advantageously, the main computer program C4 is configured to categorize the measurement data D1 temporally in vertical single jumps or vertical multiple jumps. The main computer program C4 is configured to determine a jumping force for each of the vertical single jumps or vertical multiple jumps. The main computer program C4 is configured to average the jumping force to obtain a mean jumping force value for a plurality of vertical single jumps or vertical multiple jumps. The mean value of the jumping force is referred to as the jumping force information Z3.

The main computer program C4 is configured to multiply the mean jumping force value by the mean velocity value to calculate a mean performance value. The mean performance value is referred to as the jumping performance information Z4.

The main computer program C4 is configured to calculate a jumping height by integrating the measurement data D1 twice over time. Advantageously, the main computer program C4 is configured to categorize the measurement data D1 temporally in vertical single jumps or vertical multiple jumps. The main computer program C4 is configured to determine a jumping height for each of the vertical single jumps or vertical multiple jumps. The main computer program C4 is configured to determine a mean jumping height value for a plurality of vertical single jumps or vertical multiple jumps. The mean value of the jumping height is referred to as the jumping height information Z5.

As schematically shown in FIG. 2, the main computer program C4 is configured to store in the main storage medium M4, performance data D10 that are calculated by the main computer program C4.

As schematically shown in FIG. 7, a great number of empirically determined other athletes data D11 are stored electronically in the main storage medium M4. For example, more than one million other athletes data D11 may be stored. The other athletes data D11 comprise at least one information of the following listed schematically in FIG. 7:
A third other athletes information AA3 is a gender of other athletes.
A fourth other athletes information AA4 is an age of other athletes.
A fifth other athletes information AA5 is a body size of other athletes.
A sixth other athletes information AA6 is a weight of other athletes.
A seventh other athletes information AA7 is a type of sport of other athletes.
An eighth other athletes information AA8 is a freely selectable sports characteristic of other athletes, such as “striker”, “left-footed player”, “junior”, and the like.

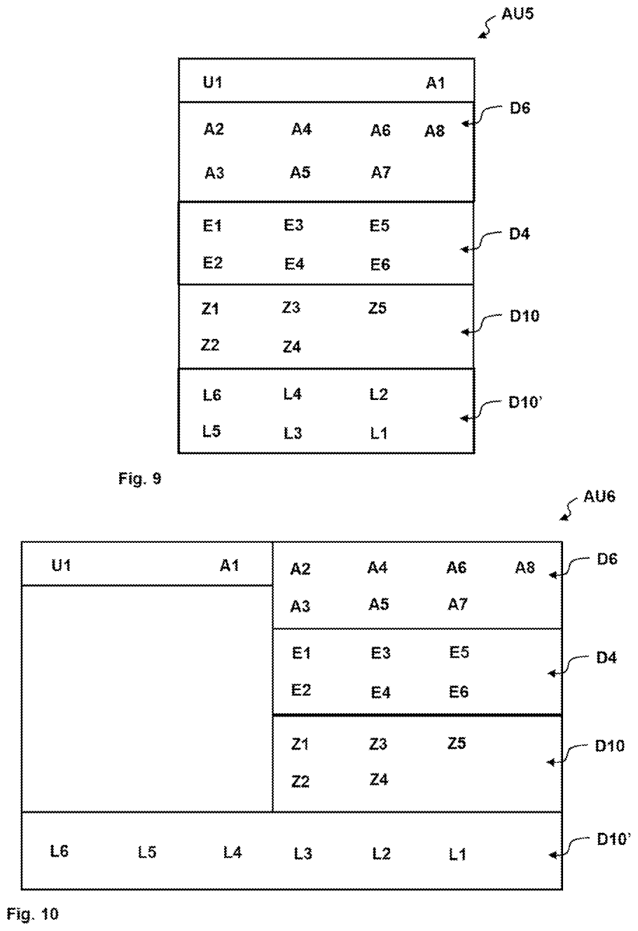

As schematically shown in FIG. 7, in the thirty-second sub-step XXXII, the main computer program C4 is configured to filter the other athletes data D11 against the athlete data D6. The athlete data D6 include at least one information of the following listed schematically in FIG. 5 and FIG. 9:
A third athlete information A3 is a gender of athlete A.
A fourth athlete information A4 is an age of athlete A.
A fifth athlete information A5 is a body size of athlete A.
A sixth athlete information A6 is a weight of athlete A.
A seventh athlete information A7 is a type of sport of athlete A.
An eighth athlete information A8 is a freely selectable sports characteristic of athlete A.

As schematically shown in FIG. 7, from the other athletes data D11, the main computer program C4 is configured to determine comparable other athletes data D12. Comparable other athletes data D12 meet at least one of the following criteria:
That the third other athletes information AA3 corresponds to the third athlete information A3. The gender of the third other athletes information AA3 must match the gender of the third athlete information A3.
That the fourth other athletes information AA4 corresponds to the fourth athlete information A4. The age of the fourth other athletes information AA4 corresponds to the age of the fourth athlete information A4 within an age range of +/−2 years, for example.
That the fifth other athletes information AA5 corresponds to the fifth athlete information A5. The body size of the fifth other athletes information AA5 corresponds to the body size of the fifth athlete information A5 within a size range of +/−5 cm, for example.
That the sixth other athletes information AA6 corresponds to the sixth athlete information A6. The weight of the sixth other athletes information AA6 corresponds to the weight of the sixth athlete information A6 within a weight range of +/−2 kg, for example.
That the seventh other athletes information AA7 corresponds to the seventh athlete information A7. The type of sport of the seventh other athletes information AA7 must correspond to the type of sport of the seventh athlete information A7.
That the eighth other athletes information AA8 corresponds to the eighth athlete information A8. The freely selectable sports characteristic of the eighth other athletes information AA8 must correspond to the freely selectable sports characteristic of the eighth athlete information A8.

The result of the filtering in the thirty-second sub-step XXXII, will be, for example, that several hundred comparable other athletes data D12 are remaining from the more than one million other athletes data D11.

The comparable other athletes data D12 include at least one of the following comparable other athletes information F1-F4 schematically represented in FIG. 7:
A jumping force information F1 of the other athletes.
A jumping velocity information F2 of the other athletes.
A jumping performance information F3 of the other athletes.
A jumping force information F1 regarding a difference in jumping force of the two lower limbs of the other athletes. For example, it is not unusual for a difference of 4% to exist in jumping force between each of the two lower limbs of an athlete.

As schematically shown in FIG. 7, in the thirty-third sub-step XXXIII, the main computer program C4 is configured to determine expert data D4 for the performance data D10. For this purpose, the main computer program C4 is configured to compare the performance data D10 of athlete A to the comparable other athletes data D12. The results of the comparison are the following expert data D4:

Performance data D10 from the jumping force information Z3 of athlete A are compared to the comparable other athletes data D12 of a jumping force information F1 of the other athletes, and the result of the comparison is the determination of expert data D4 from a first expert information E1 regarding a potentially possible jumping force of athlete A. The first expert information E1 schematically listed in FIG. 9, FIG. 10 and FIG. 13 provides information as to how closely the jumping force information Z3 of athlete A matches the jumping force information F1 of the other athletes.

Performance data D10 from athlete A's jumping velocity information Z2 are compared to comparable other athletes data D12 from a jumping velocity information F2 of the other athletes, and the result of the comparison is the determination of expert data D4 from a second expert information E2 regarding a potentially possible jumping velocity of athlete A. The second expert information E2 schematically listed in FIG. 9, FIG. 10 and FIG. 13 provides information as to how closely the jumping velocity information Z2 of athlete A matches the jumping velocity information F2 of the other athletes.

Performance data D10 from the jumping performance information Z4 of athlete A are compared to comparable other athletes data D12 from a jumping performance information F3 of the other athletes, and the result of the comparison is the determination of expert data D4 from a third expert information E3 regarding a potentially possible jumping performance of athlete A. The third expert information E3 schematically listed in FIG. 9, FIG. 10 and FIG. 13 provides information as to how closely the jumping performance information Z4 of athlete A matches the jumping performance information F3 of the other athletes.

Performance data D1 from the jumping force information Z3 of athlete A are compared to comparable other athletes data D12 from a jumping force information F4 with respect to a difference in the jumping force of the two lower limbs of the other athletes, and the result of the comparison is the determination of expert data D4 from a fourth expert information E4 regarding a difference in the jumping force of the two lower limbs of athlete A. The fourth expert information E4 schematically listed in FIG. 9, FIG. 10 and FIG. 13 provides information as to how much the difference in the jumping force of the two lower limbs of athlete A differs from the difference in the jumping force of the two lower limbs of the other athletes.

As schematically shown in FIG. 7, in the thirty-fourth sub-step XXXIV, the main computer program C4 is configured to determine further expert data D4 for the performance data D10. For this purpose, the main storage medium M4 is configured to store biometrical data D13 of an athlete and medical data D14 stating a future risk of injury of an athlete.

The biometrical data D13 are the result of biometrical model calculations. The biometrical data D13 include at least one biometrical information B1-B3 of the following:
A maximum possible jumping force information B1 of an athlete.
A maximum possible jumping velocity information B2 of an athlete.
A maximum possible jumping performance information B3 of an athlete.

The medical data D14 with respect to a future injury risk of an athlete take into account a difference in jumping force of the two lower limbs of an athlete. The reason is that if the difference in jumping force of the two lower limbs of an athlete is too high, for example more than 8%, then this difference would involve a future risk of injury for the athlete such as a torn ligament, a torn muscle, and the like.

The main computer program C4 is configured to compare performance data D10 of athlete A to the biometrical data D13 and medical data D14. The results of the comparisons are the following expert data D4 listed schematically in FIG. 9, FIG. 10 and FIG. 13:
Performance data D10 from a jumping force information Z3 of athlete A are related to performance data D10 from a jumping velocity information Z2 of athlete A to calculate performance data 10 from a jumping force-jumping velocity information Z6 of athlete A. Biometrical data D13 from a maximum possible jumping force information B1 of an athlete are related to biometrical data D13 from a maximum possible jumping velocity information B2 of an athlete to calculate biometrical data 13 from a jumping force-jumping velocity information B4 of an athlete. Thereafter, the performance data 10 from the jumping force-jumping velocity information Z6 of athlete A is compared to the biometrical data D13 from the jumping force-jumping velocity information B4 of an athlete and the result of the comparison is the determination of expert data D4 from a fifth expert information E5 about a jumping force-jumping velocity relationship of athlete A. The fifth expert information E5 provides information as to how uniformly the jumping force information Z3 and the jumping velocity information Z2 of athlete A are developed.
The expert data D4 from the fourth expert information E4 regarding a difference in jumping force of the two lower limbs of athlete A are compared to the medical data D14 and the result of the comparison is the determination of expert data D4 from a sixth expert information E6 about a future risk of injury of athlete A.

The determined performance data D10 and the determined expert data D4 are stored in the main storage medium M4 of the data processing system 4.

Performance data that were received in the past by the data processing system 4 via the main interface S4 and are stored in the main storage medium M4 as the historical performance data D10′ comprise historical performance information L1 to L6 listed schematically in FIG. 9, FIG. 10 and FIG. 13. The historical performance information L1 to L6 includes a performance information about historical jumping exercises such as a historical determined jumping acceleration information, a historical determined jumping velocity information, a historical determined jumping force information, a historical determined jumping performance information, and a historical determined jumping height information. In the sense of the present invention, the adjective “historical” means that the performance information described in this way refers to the past. This means that the performance information L1 to L6 was relevant in the past.

The historical performance information L1 to L6 is an alphanumeric string listed schematically in FIG. 9, FIG. 10 and FIG. 13, a numerical value, a graph, etc. Examples of historical performance information L1 to L6 are:
A first historical performance information L1 comprises information regarding the last jumping exercises that were performed (e.g. one week ago).
A second historical performance information L2 comprises information regarding the last but one jumping exercises that were performed (e.g. two weeks ago).
A historical third performance information L3 comprises information regarding the last but two jumping exercises that were performed (e.g. three weeks ago).
A historical fourth performance information L4 includes information regarding the last but three jumping exercises that were performed (e.g. four weeks ago).
A historical fifth performance information L5 includes information regarding the last but four jumping exercises that were performed (e.g. five weeks ago).
A historical sixth performance information L6 includes information regarding the last but five jumping exercises the were executed (e.g. six weeks ago).

### Fourth Step IV

In the fourth step IV schematically shown in FIG. 1, the performance diagnostics is acknowledged by the athlete A and/or the supervisor B. The performance diagnostics is acknowledged by the athlete A at the first computer means 5 schematically shown in FIG. 2. The performance diagnostics is acknowledged by the supervisor B at the second computer means 6 schematically shown in FIG. 2. FIG. 8 is a detailed representation of the fourth step IV comprising a forty-first sub-step XLI and a forty-second sub-step XLII.

As schematically shown in FIG. 8, in the forty-first sub-step XLI, the first computer means 5 receives athlete data D6, performance data D10, historical performance data D10′ and expert data D4 via the first interface S5. As schematically shown in FIG. 8, in the forty-first sub-step XLI, the second computer means 6 receives athlete data D6, performance data D10, historical performance data D10′ and expert data D4 via the second interface S6.

As schematically shown in FIG. 9, the first computer program C5 is configured to read the athlete data D6, the performance data D10, the historical performance data D10′, and the expert data D4 and to output these data on the first output means AU5 of the first computer means 5. The entirety of the athlete data D6 is output as the athlete information A1 to A8. Performance data D10 are output as the performance information Z1 to Z5. The entirety of the historical performance data D10′ is output as the historical performance information L1 to L6. Furthermore, the entirety of the expert data D4 is output as the expert information E1 to E6.

Referring to the schematic representation of FIG. 10, the second computer program C6 is configured to read the athlete data D6, the performance data D10, the historical performance data D10′, and the expert data D4 and to output these data on the second output means AU6 of the second computer means 6. The entirety of the athlete data D6 is output as the athlete information A1 to A8. The performance data D10 are output as the performance information Z1 to Z5. The entirety of the historical performance data D10′ is output as the historical performance information L1 to L6. Furthermore, the entirety of the expert data D4 is output as the expert information E1 to E6.

Thus, the performance information Z1 to Z5 is presented to the athlete A and/or the supervisor B together with an expert information E1 to E6. The athlete A and/or the coach B is then may use the expert information E1 to E6 to interpret the performance information Z1 to Z5.

FIG. 11 is an example of a graphical representation of the fifth expert information E5 that refers to the jumping force-jumping velocity relationship of athlete A shown on the output means AU5, AU6 of the first or second computer means 5, 6, respectively. The jumping velocity information Z2 of athlete A is plotted on the abscissa of the graph, and the jumping force information Z3 of athlete A is plotted on the ordinate of the graph. Athlete A's jumping force-jumping velocity information Z6 is plotted as a solid line. The jumping force-jumping velocity information B4 of an athlete is plotted as a dashed line.

In the example shown in FIG. 11, the comparison of the jumping force-jumping velocity information Z6 of athlete A to the jumping force-jumping velocity information B4 of an athlete provides the fifth expert information E5 stating that the jumping force-jumping velocity information Z2 of athlete A is developed too weakly, while the jumping force information Z3 of athlete A is developed too strongly. Thus, the fifth expert information E5 recommends to athlete A and to coach B, the need to enhance the velocity portion of the jumping exercises and to reduce the force portion of the jumping exercises.

In the forty-second sub-step XLII schematically shown in FIG. 8, the supervisor B and the athlete A discuss the execution of jumping exercises to be performed on a new exercise date U1. The result of this communication is new exercise data D5 comprising at least an exercise information U1 to U11. The new exercise data D5 take into account that the supervisor B and the athlete A have taken notice of the performance information Z1 to Z5 and the expert information E1 to E6. The exercise data D5 are stored in the first storage medium M5 of the first computer means 5 and in the second storage medium M6 of the second computer means 6. The new exercise data D5 are a selection of the stored exercise data D5 containing information regarding jumping exercises, which jumping exercises are to be performed by athlete A on the new exercise date U1.

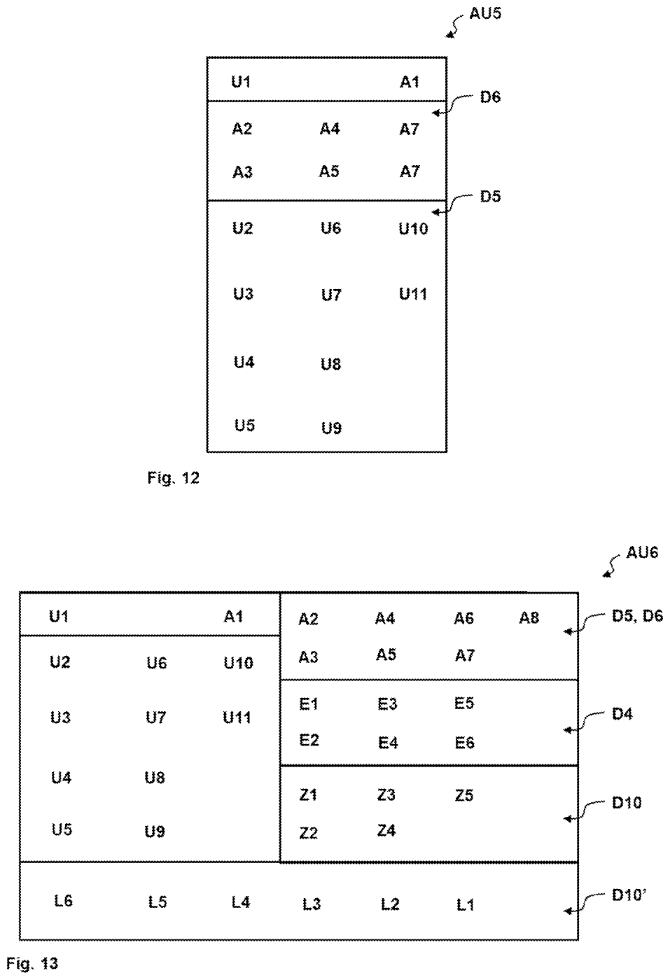

FIG. 12 is a schematic representation of the athlete data D6 and the exercise data D5 that are output by the first computer program C5 on the first output means AU5 of the first computer means 5. The entirety of the athlete data D6 is output as the athlete information A1 to A8. The entirety of the exercise data D5 is output as the exercise information U1 to Ulf.

FIG. 13 is a schematic representation of the athlete data D6, the new exercise data D5, the performance data D10, the expert data D4, and the historical performance data D10′ that are output by the second computer program C6 on the second output means AU6 of the second computer means 6. The entirety of the athlete data D6 is output as the athlete information A1 to A8. The entirety of the new exercise data D5 is output as the exercise information U1 to Ulf. The performance data D10 are output as the performance information Z1 to Z5. The entirety of the historical performance data D10′ is output as the historical performance information L1 to L6.

Having been informed by the disclosure herein of the present invention, those skilled in the art are empowered further with knowledge of a wide variety of variations of the examples. For example, the data processing system and the first computer means may be identical. In this example, there is no separate data processing system but only a first computer means. The first computer means comprises at least a first storage medium for digital data, and the main computer program is loaded into the first processor and is executed by the first processor. The executed main computer program causes the first processor to execute the third step of the method.

### LIST OF REFERENCE NUMERALS

1 measuring platform
2 camera
3 data transmission means
4 data processing system
5 first computer means
6 second computer means
10 device
A athlete
A1 bis A8 athlete information
AA3 bis AA8 other athletes information
AU4-AU6 output
B supervisor/coach
B1-B4 biometrical information
BS image sensor
C4-C6 computer program
D1 measurement data
D2 image data
D3 identification data
D4 expert data
D5 exercise data
D6 athlete data
D7 exercise time data
D8, D8′, D8″ start data
D9, D9′, D9″ stop data
D10, D10′ performance data
D11 other athletes data
D12 comparable other athletes data
D13 biometrical data
D14 medical data
E developer
E1 bis E6 expert information
E14-E16 input means
F1-F4 comparable other athletes information
KS bis KS′″ force sensor
L1 bis L6 historical performance information
M4-M6 storage medium
P1, P2, P4-P6 processor
S1, S2, S4-S6 interface
U1 bis U11 exercise information
I bis IV steps
XI bis XIII first sub-steps)
XXI bis XXIII second sub-steps
XXIa bis XXIc twenty-first alternative steps)
XXIIa, XXIIb twenty-second alternative steps
XXIIIa bis XXIIIf twenty-third alternative steps
XXXI bis XXXIV third sub-steps
XLI, XLII fourth sub-steps
Z1 bis Z6 performance information

What is claimed is:

1. A method for measuring the force exerted by an athlete exercising on a measuring platform, the method comprising the following steps:
exercise data for jumping exercises are generated by a data processing system that sends the exercise data to a first computer means;
the first computer means outputs the exercise data to the athlete to instruct the athlete how to perform jumping exercises according to the exercise data;
as the athlete performs the jumping exercises on the measuring platform, the measuring platform is measuring a jumping force of said jumping exercises on the measuring platform and generating measurement data for the measured jumping force;
the measuring platform is transmitting the generated measurement data to the data processing system;
the data processing system evaluates the measurement data and generates performance data from the evaluation of the measurement data;
the data processing system determines expert data for the performance data, and transmits the performance data and the expert data to at least one of the following: the first computer means and a second computer means; and
wherein the data processing system outputs the performance data and the expert data on at least one of the following: the first computer means and the second computer means.

2. The method according to claim 1, wherein the first computer means is used to identify the measuring platform and generate identification data comprising an identification number of the identified measuring platform and an identification number of the first computer means, and the identification data are sent from the first computer means to the data processing system; wherein the measuring platform is uniquely identified by the data processing system by means of the identification number of the measuring platform; wherein the data processing system uses the identification number of the first computer means to uniquely identify the athlete; and wherein the data processing system assigns to the athlete the measurement data generated by the measuring platform in accordance with the identification data.

3. The method according to claim 1, wherein the first computer means in a proximity of the measuring platform generates identification data comprising an identification number of the first computer means and transmits the identification data to the data processing system; wherein by means of the identification number of the first computer means, the data processing system uniquely identifies the athlete; wherein based on the exercise data outputted to the athlete, the data processing system allocates an exercise time to the athlete; wherein the data processing system sends the allocated exercise time to a second computer means as exercise time data; wherein the exercise time data are output as the exercise time to the athlete on the first computer means; wherein the athlete gets onto the measuring platform at the allocated exercise time; and wherein the data processing system assigns to the athlete the measurement data generated by the measuring platform and received directly, in terms of time, after the allocated exercise time are allocated to the athlete by the data processing system.

4. The method according to claim 1, wherein the first computer means receives a start signal that is entered by athlete into the first computer means; wherein the first computer means sends the start signal to the data processing system, which generates start data that includes the start signal and a stop signal; wherein the data processing system uniquely identifies the athlete by means of the start data; and wherein the data processing system receives the measurement data after, in terms of time, the data processing system receives the start data and identifies the athlete associated with the start data.

5. The method according to claim 1, wherein the exercise data is outputted by the first computer means to the athlete in the form of images by captured by a camera and image data are generated for the images captured by the camera; and wherein said image data are sent from the camera to the data processing system.

6. The method according to claim 1, wherein the generation of performance data includes carrying out at least one of the following determinations:
measurement data are divided by a weight of the athlete and performance data are determined from a jumping acceleration information;
the measurement data are integrated once over time and performance data are determined from a jumping velocity information;
the measurement data are averaged and performance data are determined from a jumping force information;
the measurement data are integrated once over time and performance data are determined from a jumping velocity information, wherein the measurement data are averaged and performance data are determined from a jumping force information, and wherein the jumping force information is multiplied by the jumping velocity information and performance data are determined from a jumping performance information; and
the measurement data are integrated twice over time and performance data are obtained from a jumping height information.

7. The method according to claim 6, wherein the performance data are output on a first output means of the first computer means as at least one performance information of the following:
jumping acceleration information,
jumping velocity information,
jumping force information,
jumping performance information, and
jumping height information.

8. The method according to claim 6, wherein the performance data are output on a second output means of the second computer means as at least one performance information of the following:
jumping acceleration information,
jumping velocity information,
jumping force information,
jumping performance information, and
jumping height information.

9. The method according to claim 1, wherein the athlete data include at least one information of the following:
a gender of the athlete;
an age of the athlete;
a body size of the athlete;
a weight of the athlete;
a type of sport of the athlete; and
a freely selectable sports characteristic of the athlete;
wherein the athlete data include at least one of the following information about other athletes:
a gender of other athletes;
an age of other athletes;
a body size of other athletes;
a weight of other athletes;
a type of sport of other athletes; and
a freely selectable sports characteristic of other athletes;
wherein the information about other athletes is filtered to obtain comparable other athletes information, which comparable other athletes information meets at least one of the following criteria:
the gender of the other athletes corresponds to the gender of the athlete;
the age of the other athletes corresponds to the age of the athlete;
the body size of the other athletes corresponds to the body size of the athlete;
the weight of the other athletes corresponds to the weight of the athlete;
the type of sport of the other athletes corresponds to the type of sport of the athlete; and
the freely selectable sports characteristic of the other athletes corresponds to the freely selectable sports characteristic of the athlete information.

10. The method according to claim 9, wherein the comparable other athletes information includes a jumping force information of the other athletes; wherein the performance data determined from a jumping force information are compared to the comparable other athletes information from a jumping force information of the other athletes; and wherein expert data are generated from a first expert information with respect to a potentially possible jumping force of the athlete.

11. The method according to claim 9, wherein the comparable other athletes information includes a jumping velocity information of the other athletes; wherein the performance data is determined from a jumping velocity information of the athlete and are compared to the comparable other athletes information from a jumping velocity information of the other athletes; and wherein expert data are determined from a second expert information with respect to a potentially possible jumping velocity of the athlete.

12. The method according to claim 9, wherein the comparable other information includes jumping performance information of the other athletes; wherein the performance data are determined from a jumping performance information of the athlete and are compared to the comparable other athletes information from a jumping performance information of the other athletes; and wherein expert data are determined from a third expert information with respect to a potentially possible jumping performance of the athlete.

13. The method according to claim 9, wherein the comparable other athletes information includes a jumping force information regarding a difference in the jumping force of the two lower limbs of the other athletes; wherein the performance data are determined from a jumping force information of the athlete and are compared to the comparable other athletes information from a jumping force information regarding a difference in the jumping force of the two lower limbs of the other athletes; and wherein expert data from a fourth expert information regarding a difference in the jumping force of the two lower limbs of the athlete are determined.

14. The method according to claim 9, wherein the performance data are determined from a jumping force information of the athlete and are related to performance data from a jumping velocity information of the athlete and performance data are calculated from a jumping force-jumping velocity information of the athlete; wherein biometrical data of a maximum possible jumping force information of the athlete and biometrical data of a maximum possible jumping velocity information of the athlete are provided; wherein the biometrical data of a maximum possible jumping force information of the athlete are related to the biometrical data of a maximum possible jumping velocity information of the athlete and biometrical data of a jumping force-jumping velocity information of the athlete are calculated; and wherein the performance data from the jumping force-jumping velocity information of the athlete are compared to the biometrical data from the jumping force-jumping velocity information of the athlete; and wherein expert data are determined from a fifth expert information with respect to a jumping force-jumping velocity relationship of the athlete.

15. The method according to claim 13, wherein medical data regarding a future risk of injury due to a difference in the jumping force of the two lower limbs of the athlete are provided; wherein the expert data of the fourth expert information regarding a difference in the jumping force of the two lower limbs of the athlete are compared to said medical data; and wherein expert data are obtained from a sixth expert information about a future risk of injury of the athlete.

16. The method according to claim 9, wherein expert data are outputted as the expert information on a first output means of the first computer means and/or on a second output means of the second computer means.

17. A device for carrying out a method for measuring the force exerted on a platform supporting an exercising athlete, the device comprising:
a measuring platform that includes a measuring platform processor, which is configured to generate measurement data for jumping exercises carried out on the measuring platform;
a data processing system configured for storing and transmitting jumping exercises to be carried out on the measuring platform;
a data transmission means;
a first computer means configured in communication with the data processing system via the data transmission means and configured for receiving jumping exercises transmitted from the data processing system;
wherein the measuring platform is configured to transmit the measurement data to the data processing system via the data transmission means;
wherein the data processing system includes a main processor that is configured for evaluation of the measurement data and generation of performance data from the evaluation of the measurement data and for determination of expert data for the performance data;
a second computer means configured for presenting the performance data and the expert data; and
wherein the data transmission means is configured to transmit the performance data and the expert data via the data transmission means to at least one of the following: the first computer means and the second computer means.
